(19) United States
(12) Patent Application Publication
(10) Pub. No.: US 2025/0134373 A1
(43) Pub. Date: May 1, 2025

(54) MEASUREMENT DEVICE AND CONTROL RECORDING MEDIUM

(71) Applicant: OMRON HEALTHCARE Co., Ltd., Kyoto (JP)

(72) Inventors: Nozomi NAKAWAKI, Kyoto (JP); Nobuo KUBO, Kyoto (JP)

(21) Appl. No.: 18/990,534

(22) Filed: Dec. 20, 2024

Related U.S. Application Data
(63) Continuation of application No. PCT/JP2023/017229, filed on May 8, 2023.

(30) Foreign Application Priority Data
Sep. 16, 2022 (JP) 2022-148006

(51) Int. Cl. A61B 5/00; H04W 4/38

(52) U.S. Cl. CPC A61B 5/002; H04W 4/38

(57) ABSTRACT

To provide a measurement device and a control program that can perform data transfer with a plurality of applications in one information terminal. A data storage unit of a measurement device stores a first identifier for identifying the measurement device in wireless communication by a wireless communication unit and a second identifier for identifying the measurement device in the wireless communication by the wireless communication unit. A controller of the measurement device performs a first control that causes the wireless communication unit to perform the wireless communication with a first application using the first identifier. The controller also performs a second control that causes the wireless communication unit to execute the wireless communication with a second application using the second identifier.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/017229, filed May 8, 2023, which application claims priority to Japanese Patent Application No. 2022-148006, filed Sep. 16, 2022, which applications are incorporated herein by reference in their entireties.

### TECHNICAL FIELD

The present invention relates to a measurement device and a control program.

### BACKGROUND ART

In the related art, a known data collection method is configured such that vital data measured by a wearable device such as a wristwatch-type device is transmitted to an electronic terminal such as a smart phone or a personal computer using near-field wireless communication such as Bluetooth (registered trademark), and the electronic terminal that has received the vital data transmits the vital data to a server via an Internet connection (Patent Document 1). As a method for near-field wireless communication, Bluetooth Low Energy (BLE; registered trademark) having a communication mode with low power consumption is also known in addition to Bluetooth.

### CITATION LIST

### Patent Literature

Patent Document 1: JP 2015-126804 A

### SUMMARY OF INVENTION

### Technical Problem

In recent years, there has been a need to manage data measured by a measurement device such as a blood pressure monitor equipped with a module for near-field wireless communication such as Bluetooth or BLE by using a plurality of applications installed on one information terminal. At this time, if data cannot be shared among the plurality of applications, each of the plurality of applications needs to acquire the data from the measurement device. When the measurement device starts advertisement for near-field wireless communication, the plurality of applications simultaneously start connection operations. However, there may be a situation in which only an application that is successfully connected first can acquire the data and the other applications cannot acquire the data.

In one aspect, the present invention has been made in view of such circumstances, and an object thereof is to provide a measurement device and a control program that can perform data transfer with a plurality of applications in one information terminal.

### Solution to Problem

The present invention employs the following configurations to solve the above-described problems.

(1)

A measurement device capable of performing wireless communication with an information terminal on which a first application and a second application are installed, the measurement device including:
a wireless communication unit configured to perform the wireless communication;
a storage unit configured to store a first identifier for identifying the measurement device in the wireless communication and a second identifier for identifying the measurement device in the wireless communication; and
a control unit configured to execute a first control causing the communication unit to execute the wireless communication with the first application by using the first identifier and a second control causing the communication unit to execute the wireless communication with the second application by using the second identifier.

According to (1), it is possible to perform data transfer with each of the first application and the second application which are installed on one information terminal and perform communication using the wireless communication unit in common. Accordingly, measurement data obtained through a measurement can be transferred to both the first application and the second application.

(2)

The measurement device according to (1), wherein
the wireless communication unit includes a storage area storing an identifier and performs the wireless communication by using the identifier stored in the storage area, and
the control unit executes the first control or the second control by storing the first identifier or the second identifier in the storage area.

According to (2), by storing the first identifier or the second identifier in the storage area to which the wireless communication unit refers when performing the wireless communication, it is possible to perform the wireless communication with the first application using the first identifier and the wireless communication with the second application using the second identifier without providing a plurality of wireless communication units corresponding to a plurality of applications.

(3)

The measurement device according to (1) or (2), wherein
the first identifier is an identifier of the measurement device shared through pairing between the measurement device and the first application, and
the second identifier is an identifier of the measurement device shared through pairing between the measurement device and the second application.

According to (3), by sharing the first identifier through pairing between the measurement device and the first application, and by sharing the second identifier through pairing between the measurement device and the second application, it is possible to perform the wireless communication with the first application and the second application using the first identifier and the second identifier, respectively.

(4)

The measurement device according to any one of (1) to (3), wherein the control unit alternately executes the first control and the second control.

According to (4), it is possible to reliably perform communication between the measurement device and the first application and communication between the measurement device and the second application regardless of a timing at which the first application and the second application get into a communicable state.

(5)

The measurement device according to any one of (1) to (4), wherein the control unit transmits measurement data obtained through measurement by the measurement device to the first application through the first control, and transmits the measurement data to the second application through the second control.

According to (5), the measurement data obtained by the same measurement can be transmitted to each of the first application and the second application.

(6)

The measurement device according to (5), wherein the control unit executes transmission of the measurement data to the second application through the second control upon completion of transmission of the measurement data to the first application through the first control.

According to (6), by starting the second control at a time point at which the transmission of the measurement data to the first application is completed, it is possible to quickly start the communication between the measurement device and the second application when the second application is already in a communicable state.

(7)

The measurement device according to (5) or (6), wherein the control unit causes the wireless communication unit to transmit the measurement data to the first application using a first protocol in the first control, and causes the wireless communication unit to transmit the measurement data to the second application using a second protocol different from the first protocol in the second control.

According to (7), it is possible to perform data transfer with each of the first application and the second application that perform data transfer using different protocols.

(8)

A control program for a measurement device capable of performing wireless communication with an information terminal on which a first application and a second application are installed, the measurement device including:
a wireless communication unit configured to perform the wireless communication; and
a storage unit configured to store a first identifier for identifying the measurement device in the wireless communication and a second identifier for identifying the measurement device in the wireless communication,
wherein the control program causes a processor of the measurement device to execute processing of executing a first control causing the communication unit to execute the wireless communication with the first application by using the first identifier and a second control causing the communication unit to execute the wireless communication with the second application by using the second identifier.

According to (8), it is possible to perform data transfer with each of the first application and the second application which are installed on one information terminal and perform communication using the wireless communication unit in common. Accordingly, measurement data obtained through a measurement can be transferred to both the first application and the second application.

### Advantageous Effects of Invention

According to the present invention, it is possible to provide a measurement device and a control program that can perform data transfer with a plurality of applications in one information terminal.

### BRIEF DESCRIPTION OF DRAWINGS

FIG. 1 is a diagram illustrating an information management system 10 of a first embodiment to which the present invention is applied.

FIG. 2 is a diagram illustrating a measurement state when a user measures a blood pressure value or the like using a blood pressure monitor 1A which is an example of a measurement device 1.

FIG. 3 is a diagram illustrating an example of network connection of an information terminal 5.

FIG. 4 is a block diagram illustrating a configuration of the measurement device 1.

FIG. 5 is a block diagram illustrating a configuration of the information terminal 5.

FIG. 6 is a flow chart illustrating an example of pairing processing executed by a controller 18 of the measurement device 1.

FIG. 7 is a sequence diagram illustrating a pairing operation between the measurement device 1 and a first application of the information terminal 5.

FIG. 8 is a sequence diagram illustrating a pairing operation between the measurement device 1 and a second application of the information terminal 5.

FIG. 9 is a flow chart illustrating an example of data transfer processing executed by the controller 18 of the measurement device 1.

FIG. 10 is a sequence diagram illustrating an advertisement operation of the measurement device 1.

FIG. 11 is a sequence diagram illustrating a data transfer operation between the measurement device 1 and the information terminal 5.

### DESCRIPTION OF EMBODIMENTS

Embodiments according to one aspect of the present invention will be described below based on the drawings.

### § 1 Application Example

### Information Management System 10 to Which Present Invention Is Applied

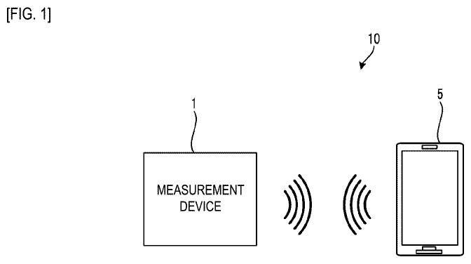

FIG. 1 is a diagram illustrating an information management system 10 of a first embodiment to which the present invention is applied. The information management system 10 includes a measurement device 1 and an information terminal 5.

The measurement device 1 includes a biological information measurement device that measures biological information such as body weight, body composition, blood pressure, pulse, heart rate, body temperature, blood glucose, or blood oxygen saturation, and an activity amount measurement device that measures an activity amount such as number of steps, walking distance, or calorie consumption. The measurement devices include a measurement sensor for measuring a measurement target amount. The measurement target amount of the measurement sensor includes biological information such as body weight, body fat percentage, blood pressure value, pulse rate, heart rate, body temperature, blood glucose value, or blood oxygen saturation, or an activity amount such as number of steps, walking distance, or calorie consumption, depending on the measurement device. The measurement device 1 is, for example, a non-wearable measurement device such as a scale, a body composition meter, a blood pressure monitor, a thermometer, or a blood glucose meter, or a wearable measurement device such as a pedometer or an activity amount meter. The non-wearable measurement device is a measurement device that is not wearable, and is, for example, a measurement device that is used in a state of being installed on the ground or a table. The wearable measurement device is a measurement device that is carried by being attached to the body of a user. The measurement device 1 transmits measured biological information or measured activity amount as measurement data of a user to the information terminal 5 by wireless communication.

The information terminal 5 stores the measurement data received from the measurement device 1 in a data storage unit in the information terminal 5. The information terminal 5 can also perform wireless communication with a device other than the measurement device 1, and stores information acquired from an external device in the data storage unit in the information terminal 5. The information terminal 5 is, for example, a terminal including a display such as a smart phone, a tablet terminal, a wearable terminal, a notebook computer, or a desktop computer. The information terminal 5 may be set to acquire measurement data from a specific measurement device 1. The specific measurement device 1 from which the measurement data is acquired may be registered in advance in the data storage unit of the information terminal 5.

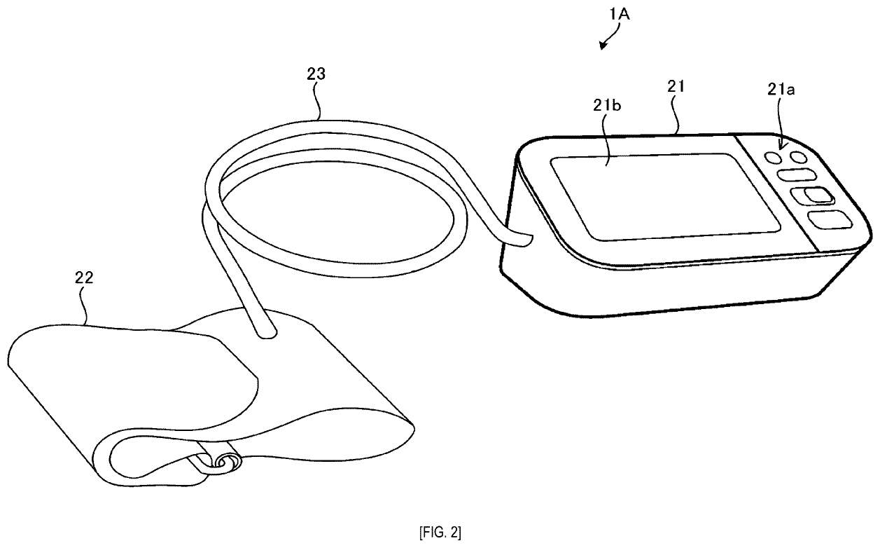

FIG. 2 is a diagram illustrating a measurement state when a user measures a blood pressure value or the like using a blood pressure monitor 1A which is an example of the measurement device 1. The blood pressure monitor 1A measures the blood pressure of a user, outputs the measurement result to the user as a numerical value, for example, and transmits the measurement result as the measurement data of the user to the information terminal 5 by wireless communication.

For example, the blood pressure monitor 1A includes a body 21, a cuff 22 that can be wrapped around an upper arm of a user, and an air tube 23 that connects the body 21 and the cuff 22. In the example of FIG. 2, the cuff 22 and the body 21 are separate bodies, but the cuff 22 may be integrated with the body 21.

The body 21 includes an operation unit 21a and a display 21b. The body 21 measures a blood pressure value of a user by, for example, an oscillometric method using the cuff 22 in response to an operation on the operation unit 21a from the user. Then, the body 21 displays information such as the measured blood pressure value and/or a pulse rate based on the measured blood pressure value on the display 21b as measurement data. The body 21 transmits the measurement data to the information terminal 5 by near-field wireless communication.

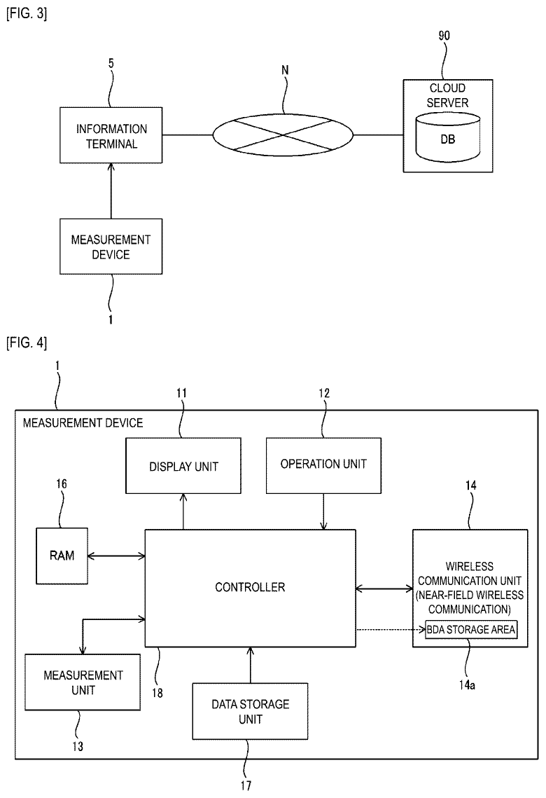

FIG. 3 is a diagram illustrating an example of network connection of the information terminal 5. As illustrated in FIG. 3, the information terminal 5 may be connected to a cloud server 90 via a wide area network N such as the Internet. The information terminal 5 may transmit the measurement data received from the measurement device 1 to the cloud server 90 via the wide area network N, and the cloud server 90 may manage the measurement data of the user as a database. The information terminal 5 may acquire the measurement data managed in the cloud server 90 via the wide area network N and use the acquired measurement data.

### § 2 Configuration Example

### Configuration of Measurement Device 1

FIG. 4 is a block diagram illustrating a configuration of the measurement device 1. The measurement device 1 includes a display unit 11 that displays various information, an operation unit 12 operated by a user, a measurement unit 13 that measures biological information, activity amount, and the like, and a wireless communication unit 14 that communicates with an external device. The measurement device 1 further includes a RAM 16 that temporarily stores information, a data storage unit 17 that stores information and programs, and a controller 18 that controls the overall operation. The controller 18 is an example of “a control unit” and “a processor” in the present invention.

The display unit 11 includes a display such as a liquid crystal display or an organic Electro-Luminescence (EL) display, for example. The operation unit 12 is a user interface such as a button or a touch panel that receives a user operation. The button includes a button physically provided at the measurement device 1 or a virtual button displayed on the display unit 11. When the measurement device 1 is the blood pressure monitor 1A, the display unit 11 corresponds to, for example, the display 21b.

The measurement unit 13 measures biological information such as body weight, body composition, blood pressure, pulse, heart rate, body temperature, blood glucose, or blood oxygen saturation, or an activity amount such as number of steps, walking distance, or calories consumption. What is measured varies depending on a measurement target of the measurement device 1. When the measurement device 1 is the blood pressure monitor 1A, the measurement unit 13 corresponds to, for example, the cuff 22 and the air tube 23.

The wireless communication unit 14 is a communication unit that performs near-field wireless communication using an antenna (not illustrated) included in the measurement device 1, for example, a BLE microcomputer for performing BLE communication in accordance with the BLE standard. The BLE communication performed by the wireless communication unit 14 is, for example, communication using 2.4 GHz frequencies. The wireless communication unit 14 operates as, for example, a peripheral node in the BLE communication.

For example, the wireless communication unit 14 as a peripheral node transmits, to an unspecified number of external devices by broadcast communication, an advertisement signal for another communication device (e.g., the information terminal 5) as a central node to perform BLE communication with the wireless communication unit 14.

In addition, the wireless communication unit 14 includes a BDA storage area 14a. The BDA storage area 14a stores a Bluetooth Device Address (BDA) which is an identifier for identifying the wireless communication unit 14 (the measurement device 1) in the BLE communication. The wireless communication unit 14 performs the BLE communication using the BDA stored in the BDA storage area 14a. The BDA stored in the BDA storage area 14a is rewritable by the controller 18.

The RAM 16 includes, for example, a semiconductor device such as a DRAM or an SRAM and temporarily stores information and serves as a work area for the controller 18.

The data storage unit 17 is a recording medium that stores parameters, a control program, measurement data, and the like necessary for implementing a predetermined processing. The data storage unit 17 includes, for example, a hard disk drive (HDD) or a semiconductor storage device (SSD).

The controller 18 executes the control program to implement the predetermined processing. In the present embodiment, for example, health management application software for the measurement device is installed in advance as the control program in the data storage unit 17, and the controller 18 implements the predetermined processing by executing the health management application software.

### Configuration of Information Terminal 5

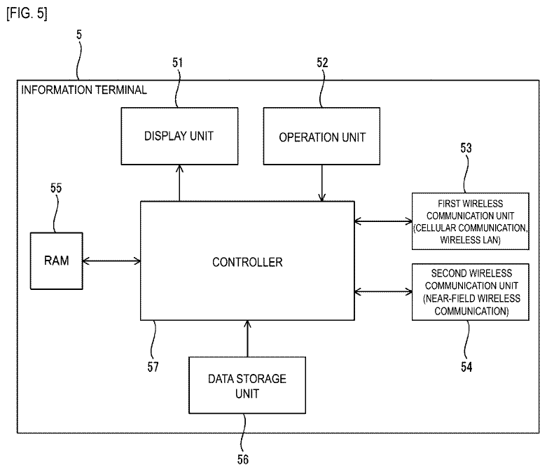

FIG. 5 is a block diagram illustrating a configuration of the information terminal 5. The information terminal 5 includes a display unit 51 that displays various information, an operation unit 52 operated by a user, and a first wireless communication unit 53 and a second wireless communication unit 54 that communicate with an external device. The information terminal 5 further includes a RAM 55 that temporarily stores information, a data storage unit 56 that stores information and programs, and a controller 57 that controls the overall operation.

The display unit 51 includes a liquid crystal display or an organic EL display, for example. The operation unit 52 is a user interface such as a button or a touch panel that receives a user operation. The button includes a button physically provided at the information terminal 5 or a virtual button displayed on the display unit 51.

The first wireless communication unit 53 is a communication unit that performs cellular communication, for example, a circuit (module) for performing communication in accordance with a standard such as 4G, 5G, or Long Term Evolution (LTE; registered trademark). In addition, the first wireless communication unit 53 is a communication unit that performs wireless LAN communication, for example, a circuit (module) for performing communication in accordance with a standard such as Wi-Fi (registered trademark). The second wireless communication unit 54 is a communication unit that performs near-field wireless communication, for example, a BLE microcomputer for performing BLE communication in accordance with the BLE standard. The wireless communication (BLE communication) performed by the second wireless communication unit 54 is, for example, communication using 2.4 GHz frequencies.

The second wireless communication unit 54 operates as, for example, a central node in the BLE communication. For example, the second wireless communication unit 54 receives an advertisement signal from the measurement device 1 by performing scanning as a central node, and performs the BLE communication with the measurement device 1 based on the received advertisement signal, thereby receiving measurement data such as biological information or an activity amount.

The RAM 55 includes, for example, a semiconductor device such as a DRAM or an SRAM and temporarily stores information and serves as a work area for the controller 57.

The data storage unit 56 is a recording medium that stores parameters, a control program, measurement data acquired from the measurement device 1, and the like necessary for implementing a predetermined processing. The data storage unit 56 includes, for example, a hard disk drive (HDD) or a semiconductor storage device (SSD). The controller 57 executes the control program stored in the data storage unit 56 to implement the predetermined processing.

At least a first application and a second application different from the first application are installed on the information terminal 5. Specifically, a program of the first application and a program of the second application are stored in the data storage unit 56, so that the controller 57 can execute the first application and the second application.

Each of the first application and the second application is a health management application including a process of performing wireless communication with the measurement device 1 via the second wireless communication unit 54 to acquire measurement data from the measurement device 1. As an example, the first application is an application that acquires measurement data from the measurement device 1 using a standardized data transfer protocol (standard protocol), and the second application is an application that acquires the measurement data from the measurement device 1 using a data transfer protocol different from the standard protocol (e.g., a unique protocol of a manufacturer of the measurement device 1). However, the first application and the second application may be applications that acquire measurement data from the measurement device 1 using the same data transfer protocol.

### § 3 Operation Example

### Pairing Processing by Controller 18 of Measurement Device 1

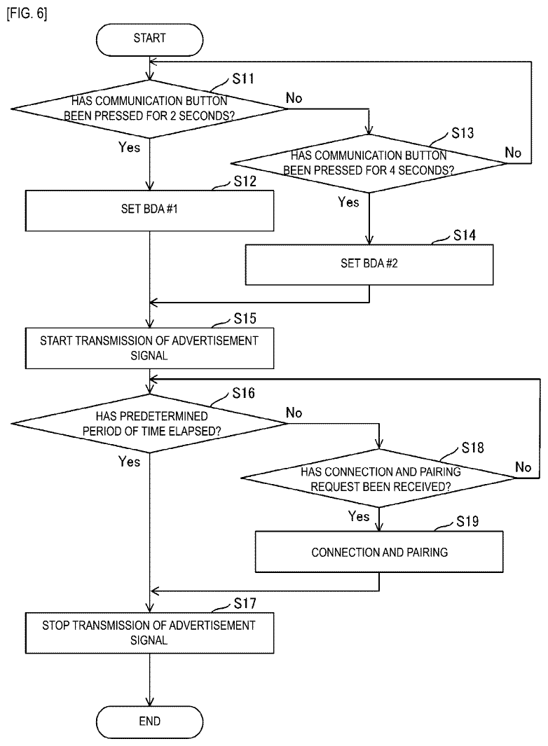

FIG. 6 is a flow chart illustrating an example of pairing processing executed by the controller 18 of the measurement device 1. The controller 18 of the measurement device 1 executes, for example, processing illustrated in FIG. 6 as the pairing processing. Here, it is assumed that a BDA #1 and a BDA #2 are stored as (configurable) writable BDAs in the BDA storage area 14a in the data storage unit 17 of the measurement device 1.

First, the controller 18 determines whether 2-second pressing of a communication button of the measurement device 1 has been received (step S11). The communication button of the measurement device 1 is included, for example, in the operation unit 12 of the measurement device 1, and is, for example, a button included in the operation unit 21a when the measurement device 1 is the blood pressure monitor 1A. The 2-second pressing of the communication button is, for example, an operation of pressing the communication button for a time period of 2 seconds or more and less than 4 seconds. That is, the 2-second pressing of the communication button is, for example, an operation of pressing the communication button for 2 seconds or more and releasing the communication button before 4 seconds elapse from the start of the pressing. When the 2-second pressing of the communication button has been received (step S11: Yes), the controller 18 sets the BDA #1,which is stored in the data storage unit 17, in the BDA storage area 14a of the wireless communication unit 14 (step S12), and proceeds to step S15.

When the 2-second pressing of the communication button has not been received in step S11, the controller 18 determines whether 4-second pressing of the communication button of the measurement device 1 has been received (step S13). The 4-second pressing of the communication button is, for example, an operation of pressing the communication button for 4 seconds or more. When the 4-second pressing of the communication button has been received (step S13: Yes), the controller 18 sets the BDA #2, which is stored in the data storage unit 17, in the BDA storage area 14a of the wireless communication unit 14 (step S14), and proceeds to step S15.

In step S15, the controller 18 controls the wireless communication unit 14 to start transmission of an advertisement signal including the BDA set in the BDA storage area 14a (step S15).

Next, the controller 18 determines whether a predetermined period of time (e.g., 10 seconds) has elapsed from the start of the transmission of the advertisement signal by step S15 (step S16). When the predetermined period of time has elapsed (step S16: Yes), the controller 18 stops the transmission of the advertisement signal started by step S15 (step S17) and ends the series of processing steps.

When the predetermined period of time has not elapsed in step S16 (step S16: No), the controller 18 determines whether a request for connection and pairing for BLE communication has been received from another communication device (e.g., the information terminal 5) (step S18).

When the request for connection and pairing for the BLE communication has been received in step S18 (step S18: Yes), the controller 18 performs connection and pairing for the BLE communication in response to the request (step S19), and proceeds to step S17. The pairing in step S19 is performed based on the BDA set in the BDA storage area 14a by step S12 or step S14. As a result, the BDA set in the BDA storage area 14a by step S12 or step S14 can be stored as the BDA of the measurement device 1 in the communication device (e.g., the information terminal 5) which is the pairing destination. If the request for connection and pairing for the BLE communication has not been received (step S18: No), the controller 18 returns to step S16.

### Pairing Operation

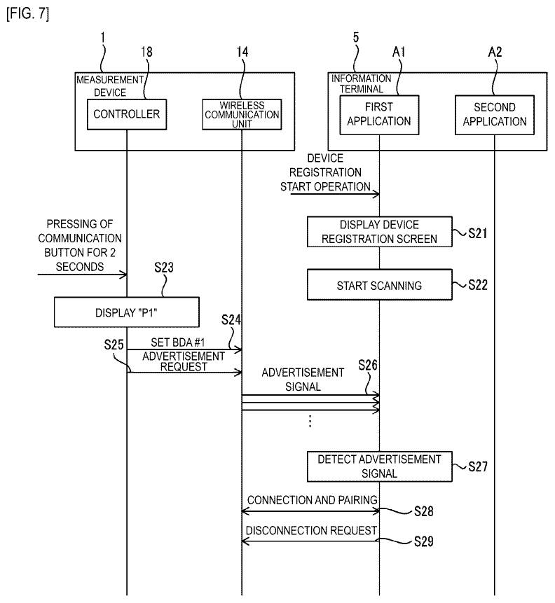

FIG. 7 is a sequence diagram illustrating a pairing operation between the measurement device 1 and a first application of the information terminal 5. Here, the above-described first application is referred to as a first application A1, and the above-described second application is referred to as a second application A2.

First, it is assumed that a user has performed a device registration start operation for the first application A1 of the information terminal 5. In response to this, the first application A1 controls the display unit 51 to display a device registration screen (step S21). On the device registration screen, for example, a message prompting the user to press the communication button of the measurement device 1 for 2 seconds or 4 seconds is displayed. In addition, the first application A1 controls the second wireless communication unit 54 to start scanning for receiving an advertisement signal from a peripheral node (step S22).

Next, it is assumed that the user has pressed the communication button of the measurement device 1 for 2 seconds. In response to this, the controller 18 of the measurement device 1 displays “P1” on the display unit 11 of the measurement device 1 (step S23). This “P1” indicates that the BDA #1 out of the BDA #1 and the BDA #2 stored in the data storage unit 17 is used as a BDA for the BLE communication. The controller 18 sets the BDA #1 in the BDA storage area 14a of the wireless communication unit 14 (step S24).

Next, the controller 18 makes an advertisement request to the wireless communication unit 14 (step S25). Then, the wireless communication unit 14 starts transmission of an advertisement signal in response to the advertisement request from the controller 18 (step S26). The advertisement signal, the transmission of which is started by step S26, includes, as the BDA, the BDA #1 set in the BDA storage area S24 by step 14a.

Next, the first application A1 detects the advertisement signal, the transmission of which is started by step S26, by the scanning started by step S22 (step S27). Then, the first application A1 controls the second wireless communication unit 54 to perform connection and pairing for the BLE communication with the measurement device 1 using the BDA #1 included in the advertisement signal detected by step S27 (step S28). By step S28, the measurement device 1 and the information terminal 5 exchange their identification information (including the BDA #1 of the measurement device 1) with each other to get into a state in which they have recognized each other (communicable state).

Next, the first application A1 controls the second wireless communication unit 54 to make a disconnection request to the measurement device 1 (step S29). As a result, the connection of the BLE communication between the measurement device 1 and the information terminal 5 is once disconnected, and the series of pairing operation steps ends.

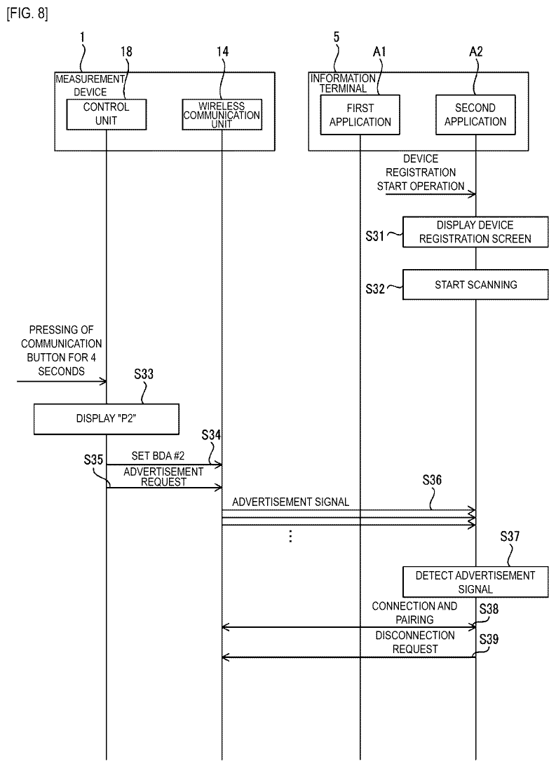

FIG. 8 is a sequence diagram illustrating a pairing operation between the measurement device 1 and the second application of the information terminal 5.

First, it is assumed that a user has performed a device registration start operation for the second application A2 of the information terminal 5. In response to this, the second application A2 executes the processing of steps S31 and S32. The processing of steps S31 and S32 is similar to the processing of steps S21 and S22 executed by the first application A1 illustrated in FIG. 7.

Next, it is assumed that the user has pressed the communication button of the measurement device 1 for 4 seconds. In response to this, the controller 18 of the measurement device 1 displays “P2” on the display unit 11 of the measurement device 1 (step S33). This “P2” indicates that the BDA #2 out of the BDA #1 and the BDA #2 stored in the data storage unit 17 is used as a BDA for the BLE communication. The controller 18 sets the BDA #2 in the BDA storage area 14a of the wireless communication unit 14 (step S34).

Next, the controller 18 makes an advertisement request to the wireless communication unit 14 (step S35). Then, the wireless communication unit 14 starts transmission of an advertisement signal in response to the advertisement request from the controller 18 (step S36). The advertisement signal, the transmission of which is started by step S36, includes, as the BDA, the BDA #2 set in the BDA storage area 14a by step S34.

Next, the second application A2 detects the advertisement signal, the transmission of which is started by step S36, by the scanning started by step S32 (step S37). Then, the second application A2 controls the second wireless communication unit 54 to perform connection and pairing for the BLE communication with the measurement device 1 using the BDA #2 included in the advertisement signal detected by step S37 (step S38). By step S38, the measurement device 1 and the information terminal 5 exchange their identification information (including the BDA #2 of the measurement device 1) with each other to get into a state in which they have recognized each other (communicable state).

Next, the second application A2 controls the second wireless communication unit 54 to make a disconnection request to the measurement device 1 (step S39). As a result, the connection of the BLE communication between the measurement device 1 and the information terminal 5 is once disconnected, and the series of pairing operation steps ends.

By performing the pairing operation illustrated in FIG. 7 and the pairing operation illustrated in FIG. 8, the pairing between the measurement device 1 and the first application A1 is performed using the BDA #1 of the measurement device 1, and the pairing between the measurement device 1 and the second application A2 is performed using the BDA #2 of the measurement device 1. Accordingly, the first application A1 can perform the BLE communication with the measurement device 1 using the BDA #1 of the measurement device 1, and the second application A2 can perform the BLE communication with the measurement device 1 using the BDA #2 of the measurement device 1.

### Data Transfer Processing by Controller 18 of Measurement Device 1

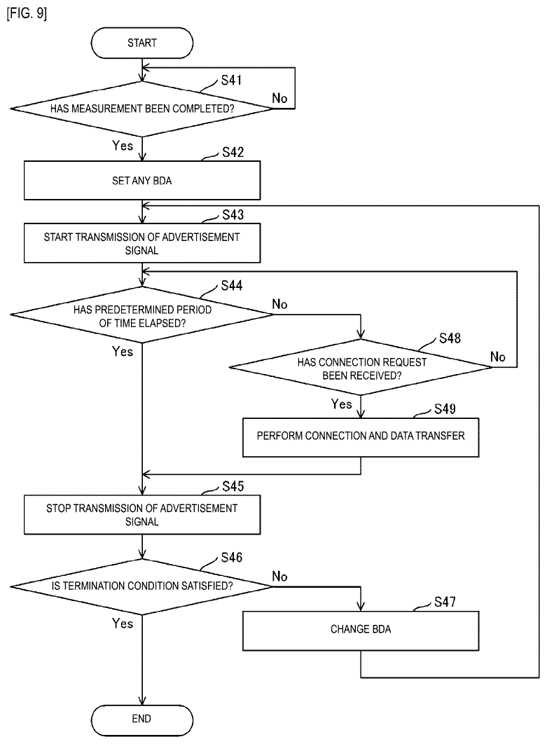

FIG. 9 is a flow chart illustrating an example of data transfer processing executed by the controller 18 of the measurement device 1. The controller 18 of the measurement device 1 executes, for example, the processing illustrated in FIG. 9 as the data transfer processing during measurement by the measurement unit 13.

First, the controller 18 determines whether the measurement by the measurement unit 13 has been completed (step S41), and waits until the measurement by the measurement unit 13 is completed (step S41: loop of No). When the measurement by the measurement unit 13 has been completed (step S41: Yes), the controller 18 sets either of the BDAs (e.g., the BDA #1), which are stored in the data storage unit 17, in the BDA storage area 14a of the wireless communication unit 14 (step S42).

Next, the controller 18 controls the wireless communication unit 14 to start transmission of an advertisement signal including the BDA set in the BDA storage area 14a (step S43). Then, the controller 18 determines whether a predetermined period of time (e.g., 10 seconds) has elapsed from the start of the transmission of the advertisement signal by step S43 (step S44). When the predetermined period of time has elapsed (step S44: Yes), the controller 18 stops the transmission of the advertisement signal started by step S43 (step S45).

Next, the controller 18 determines whether a termination condition for the data transfer processing is satisfied (step S46). The termination condition for the data transfer processing is, for example, that the advertisement signal has been transmitted N times for all the BDAs stored in the data storage unit 17. Nis a natural number equal to or greater than 1. Alternatively, the termination condition for the data transfer processing may be that data transfer has been completed for all the BDAs stored in the data storage unit 17.

In step S46, when the termination condition is not satisfied (step S46: No), the controller 18 changes the BDA to be set in the BDA storage area 14a to another BDA stored in the data storage unit 17 (step S47), and returns to step S43. Accordingly, the transmission of an advertisement signal with a changed BDA is started.

When the predetermined period of time has not elapsed in step S44 (step S44: No), the controller 18 determines whether a request for connection of the BLE communication and data transfer has been received from another communication device (e.g., the information terminal 5) (step S48). When the request for connection of the BLE communication and data transfer has not been received (step S48: No), the controller 18 returns to step S44.

When the request for connection of the BLE communication and data transfer has been received in step S48 (step S48: Yes), the controller 18 performs connection of the BLE communication and data transfer in response to the request (step S49), and proceeds to step S45. In step S49, the controller 18 transfers measurement data based on the measurement by step S41 to the connection destination (e.g., the information terminal 5).

When the termination condition is satisfied in step S46 (step S46: Yes), the controller 18 ends the series of processing steps.

### Advertisement Operation

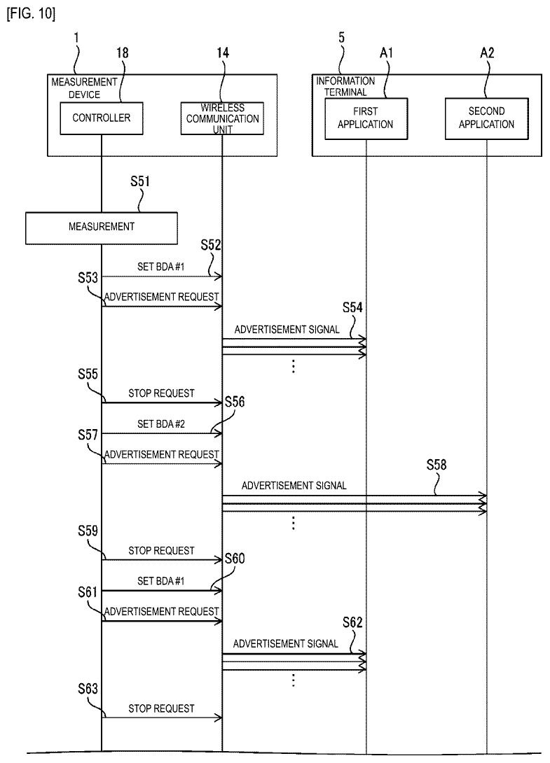

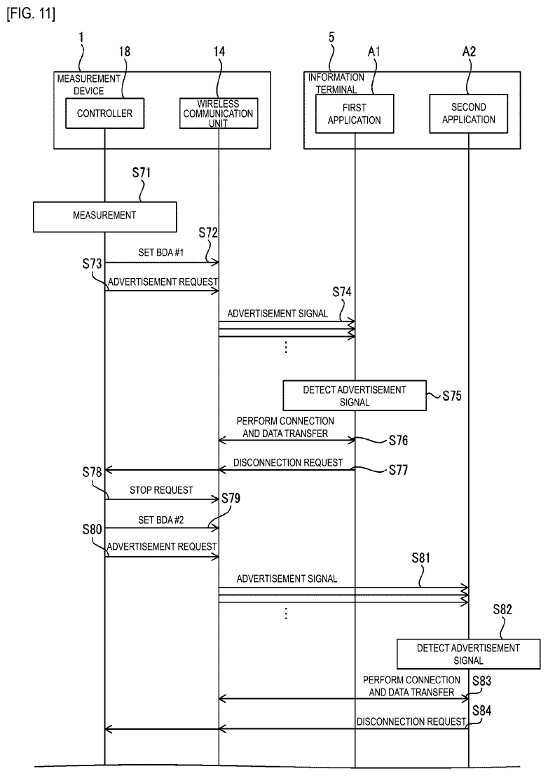

FIG. 10 is a sequence diagram illustrating an advertisement operation of the measurement device 1. First, the controller 18 controls the measurement unit 13 to perform measurement in response to an operation on the operation unit 12 from a user (step S51).

Next, the controller 18 sets the BDA #1, which is stored in the data storage unit 17, in the BDA storage area 14a of the wireless communication unit 14 (step S52). The controller 18 makes an advertisement request to the wireless communication unit 14 (step S53). Then, the wireless communication unit 14 starts transmission of an advertisement signal in response to the advertisement request from the controller 18 (step S54). The advertisement signal, the transmission of which is started by step S54, includes, as the BDA, the BDA #1 set by step S52.

Next, when a certain period of time (e.g., 10 seconds) has elapsed from the start of the transmission of the advertisement signal by steps S53 and S54, the controller 18 makes a stop request for the transmission of the advertisement signal to the wireless communication unit 14 (step S55). As a result, in response to the stop request from the controller 18, the wireless communication unit 14 stops the transmission of the advertisement signal started by step S54.

Next, the BDA #2 stored in the data storage unit 17 is set in the BDA storage area 14a of the wireless communication unit 14 (step S56). The controller 18 makes an advertisement request to the wireless communication unit 14 (step S57). Then, the wireless communication unit 14 starts transmission of an advertisement signal in response to the advertisement request from the controller 18 (step S58). The advertisement signal, the transmission of which is started by step S58, includes, as the BDA, the BDA #2 set by step S56.

Next, when a certain period of time (e.g., 10 seconds) has elapsed from the start of the transmission of the advertisement signal by steps S57 and S58, the controller 18 makes a stop request for the transmission of the advertisement signal to the wireless communication unit 14 (step S59). As a result, in response to the stop request from the controller 18, the wireless communication unit 14 stops the transmission of the advertisement signal started by step S58.

Thereafter, the controller 18 repeats processing similar to steps S52 to S59 N times. For example, steps S60 to S63 are processing similar to steps S52 to S55.

### Data Transfer Operation

FIG. 11 is a sequence diagram illustrating a data transfer operation between the measurement device 1 and the information terminal 5. It is assumed that the first application A1 and the second application A2 control the second wireless communication unit 54 in response to an operation from a user or by background processing to perform scanning for receiving an advertisement signal. At this time, the first application A1 detects an advertisement signal including the BDA #1 of the measurement device 1 acquired by the pairing operation illustrated in FIG. 7. The second application A2 detects an advertisement signal including the BDA #2 of the measurement device 1 acquired by the pairing operation illustrated in FIG. 8.

In this case, for example, the processing illustrated in FIG. 11 is executed during measurement by the measurement device 1. Steps S71 to S74 illustrated in FIG. 11 are similar to steps S51 to S54 illustrated in FIG. 10. In the example of FIG. 11, it is assumed that the first application A1 of the information terminal 5 has detected the advertisement signal including the BDA #1 the transmission of which is started by the wireless communication unit 14 by step S74 (step S75).

Next, the first application A1 controls the second wireless communication unit 54 to perform connection of the BLE communication and data transfer with the measurement device 1 using the BDA #1 included in the advertisement signal detected by step S75 (step S76). By step S76, the first application A1 receives the measurement data obtained by step S71 from the measurement device 1. Then, the first application A1 stores the received measurement data in an area accessible by the first application A1 in the data storage unit 56.

Next, the first application A1 controls the second wireless communication unit 54 to make a disconnection request to the measurement device 1 (step S77). As a result, the connection of the BLE communication between the measurement device 1 and the information terminal 5 is disconnected. At this time, the wireless communication unit 14 of the measurement device 1 may notify the controller 18 that the connection of the BLE communication with the first application A1 has been disconnected.

Then, the controller 18 proceeds to step S79 upon the elapse of a predetermined period of time (e.g., 10 seconds) from the start of the transmission of the advertisement signal by steps S73 and S74. Alternatively, the controller 18 may proceed to step S79 upon the completion of the data transfer with the first application A1.

Steps S79 to S81 illustrated in FIG. 11 are similar to steps S56 to S58 illustrated in FIG. 10. In the example of FIG. 11, it is assumed that the second application A2 of the information terminal 5 has detected the advertisement signal including the BDA #2 the transmission of which is started by the wireless communication unit 14 by step S81 (step S82).

Next, the second application A2 controls the second wireless communication unit 54 to perform connection of the BLE communication and data transfer with the measurement device 1 using the BDA #2 included in the advertisement signal detected by step S82 (step S83). By step S83, the second application A2 receives the measurement data obtained by step S71 from the measurement device 1. Then, the second application A2 stores the received measurement data in an area accessible by the second application A2 in the data storage unit 56.

Next, the second application A2 controls the second wireless communication unit 54 to make a disconnection request to the measurement device 1 (step S84). As a result, the connection of the BLE communication between the measurement device 1 and the information terminal 5 is disconnected. At this time, the wireless communication unit 14 may notify the controller 18 that the connection of the BLE communication with the second application A2 has been disconnected.

Steps S72 to S77 illustrated in FIG. 11 are an example of the first control of the present invention. Steps S79 to S84 illustrated in FIG. 11 are an example of the second control of the present invention.

As described above, according to the measurement device 1, it is possible to perform the first control for causing the wireless communication unit 14 to execute wireless communication with the first application A1 using the BDA #1 (the first identifier) and the second control for causing the wireless communication unit 14 to execute wireless communication with the second application A2 using BDA #2 (the second identifier). Accordingly, the measurement device 1 can perform data transfer with each of the first application A1 and the second application A2 which are installed on one information terminal 5 and perform communication using the second wireless communication unit 54 in common. As a result, the measurement device 1 can transfer the measurement data obtained by the measurement unit 13 to both the first application A1 and the second application A2.

For example, by transmitting an advertisement signal using the BDA #1, the measurement device 1 can communicate with only the first application A1 with which the BDA #1 is shared in advance, out of the first application A1 and the second application A2. In addition, by transmitting an advertisement signal using the BDA #2, the measurement device 1 can communicate with only the second application A2 with which the BDA #2 is shared in advance, out of the first application A1 and the second application A2.

As a result, for example, it is possible to prevent a situation in which upon completion of measurement, the first application A1 and the second application A2 simultaneously start the connection operation in response to an advertisement signal transmitted by the measurement device 1, and only the application that is successfully connected first to the measurement device 1 out of the first application A1 and the second application A2 can acquire measurement data, and the other application cannot acquire the measurement data.

In addition, by storing the BDA #1 or the BDA #2 in the BDA storage area 14a to which the wireless communication unit 14 refers when performing wireless communication, the measurement device 1 can perform wireless communication with the first application A1 using the BDA #1 and wireless communication with the second application A2 using the BDA #2 without providing a plurality of wireless communication units 14 corresponding to the first application A1 and the second application A2.

Further, by sharing the BDA #1 through pairing between the measurement device 1 and the first application A1, and by sharing the BDA #2 through pairing between the measurement device 1 and the second application A2, wireless communication can be performed with the first application A1 and the second application A2 using the BDA #1 and the BDA #2, respectively.

The measurement device 1 alternately executes the first control using the BDA #1 (e.g., steps S72 to S77 illustrated in FIG. 11) and the second control using the BDA #2 (e.g., steps S79 to S84 illustrated in FIG. 11). Accordingly, it is possible to reliably perform communication between the measurement device 1 and the first application A1 and communication between the measurement device 1 and the second application A2 regardless of a timing at which the first application A1 and the second application A2 get into a communicable state.

The measurement device 1 transmits measurement data obtained through measurement by the measurement unit 13 to the first application A1 through the first control, and similarly transmits the measurement data obtained through the measurement by the measurement unit 13 to the second application A2 through the second control. Accordingly, the measurement data obtained through the same measurement can be transmitted to each of the first application A1 and the second application A2.

The measurement device 1 may execute the transmission of the measurement data to the second application A2 through the second control upon completion of the transmission of the measurement data to the first application A1 through the first control. Accordingly, by starting the second control at a time point at which the transmission of the measurement data to the first application A1 is completed, it is possible to quickly start the communication between the measurement device 1 and the second application A2 when the second application A2 is already in a communicable state.

The measurement device 1 causes the wireless communication unit 14 to transmit the measurement data to the first application A1 using a first protocol (e.g., a standard protocol) in the first control, and causes the wireless communication unit 14 to transmit the measurement data to the second application A2 using a second protocol (e.g., a unique protocol of a manufacturer) different from the first protocol in the second control. Accordingly, it is possible to perform data transfer with each of the first application A1 and the second application A2 that perform data transfer using different protocols.

### § 4 Program

Note that the control method for the measurement device 1 explained in the above-described embodiment can be realized by a computer executing a control program prepared in advance. The control program is recorded in a computer-readable storage medium and is executed by being read from the storage medium. The control program may be provided in a form of being stored in a non-transitory storage medium such as a flash memory or may be provided via a network such as the Internet. The computer that executes the control program may be included in the measurement device 1, may be included in an electronic device such as a smart phone, a tablet terminal, or a personal computer that can communicate with the measurement device 1, or may be included in a server device that can communicate with the measurement device 1 and the electronic device.

### § 5 Modified Examples

### Modified Example of Switching Between First Control and Second Control

The configuration in which the first control and the second control are switched with the elapse of a time or the completion of data transfer as a trigger has been described. However, the first control and the second control may be switched by another trigger. For example, the operation unit 12 of the measurement device 1 (e.g., the operation unit 21a of the blood pressure monitor 1A) may be provided with an application switching operation unit that switches an application which is a communication destination, and the measurement device 1 may switch between the first control and the second control with an operation of the application switching operation unit as a trigger.

### Modified Example of Identifier

The configuration in which data transfer is performed between two applications (the first application A1 and the second application A2) using two identifiers (the BDA #1 and the BDA #2) has been described. However, a configuration in which data transfer is performed between three or more applications using three or more identifiers may be employed.

While embodiments of the present invention have been described in detail above, the foregoing description is merely illustrative of the present invention in all respects. Various modifications and variations can be made without departing from the scope of the present invention.

While various embodiments have been described above, needless to say, the present invention is not limited to such examples. It will be apparent to those skilled in the art that various changes and modifications can be made within the scope of the claims, and it should be understood that such changes and modifications also fall within the technical scope of the present invention. Further, components in the above-described embodiments may be combined as desired without departing from the gist of the invention.

Note that the present application is based on Japanese Patent Application filed on Sep. 16, 2022 (JP 2022-148006), the content of which is incorporated herein by reference.

### REFERENCE NUMERALS LIST

1 Measurement device
1A Blood pressure monitor
5 Information terminal
10 Information management system
11, 51 Display unit
12, 21a, 52 Operation unit
13 Measurement unit
14 Wireless communication unit
14a BDA storage area
16, 55 RAM
17, 56 Data storage unit
18, 57 Controller
21 Body
21b Display
22 Cuff
23 Air tube
53 First wireless communication unit
54 Second wireless communication unit
90 Cloud server
A1 First application
A2 Second application

1. A measurement device capable of performing wireless communication with an information terminal on which a first application and a second application are installed, the measurement device comprising:
a wireless communication unit configured to perform the wireless communication;
a storage unit configured to store a first identifier for identifying the measurement device in the wireless communication and a second identifier for identifying the measurement device in the wireless communication; and
a control unit configured to execute a first control causing the communication unit to execute the wireless communication with the first application by using the first identifier and a second control causing the communication unit to execute the wireless communication with the second application by using the second identifier.

2. The measurement device according to claim 1, wherein
the wireless communication unit includes a storage area storing an identifier and performs the wireless communication by using the identifier stored in the storage area, and
the control unit executes the first control or the second control by storing the first identifier or the second identifier in the storage area.

3. The measurement device according to claim 1, wherein
the first identifier is an identifier of the measurement device shared through pairing between the measurement device and the first application, and
the second identifier is an identifier of the measurement device shared through pairing between the measurement device and the second application.

4. The measurement device according to claim 1, wherein the control unit alternately executes the first control and the second control.

5. The measurement device according to claim 1, wherein the control unit transmits measurement data obtained through measurement by the measurement device to the first application through the first control, and transmits the measurement data to the second application through the second control.

6. The measurement device according to claim 5, wherein the control unit executes transmission of the measurement data to the second application through the second control upon completion of transmission of the measurement data to the first application through the first control.

7. The measurement device according to claim 5, wherein the control unit causes the wireless communication unit to transmit the measurement data to the first application using a first protocol in the first control, and causes the wireless communication unit to transmit the measurement data to the second application using a second protocol different from the first protocol in the second control.

8. A recording medium for a measurement device capable of performing wireless communication with an information terminal on which a first application and a second application are installed, the measurement device comprising:
a wireless communication unit configured to perform the wireless communication; and
a storage unit configured to store a first identifier for identifying the measurement device in the wireless communication and a second identifier for identifying the measurement device in the wireless communication,
wherein the control program causes a processor of the measurement device to execute processing of executing a first control causing the communication unit to execute the wireless communication with the first application by using the first identifier and a second control causing the communication unit to execute the wireless communication with the second application by using the second identifier.
